(19) United States
(12) Patent Application Publication
(10) Pub. No.: US 2025/0270570 A1
(43) Pub. Date: Aug. 28, 2025

(54) QUANTUM-TACTIC YEAST INGREDIENT METHOD FOR OUTBREAK MONITORING WITH RADIOWAVE OPTICS BY SATELLITE ARRAYS ON LAGRANGIAN-1

(71) Applicant: Farida Hanna Campbell, Maastricht (NL)

(72) Inventor: Farida Hanna Campbell, Maastricht (NL)

(21) Appl. No.: 18/575,741

(22) PCT Filed: Jun. 29, 2022
(86) PCT No.: PCT/EP2022/067960

§ 371 Date: Dec. 29, 2023

(30) Foreign Application Priority Data
Jul. 1, 2021 (EP) 21183136.7

(51) Int. Cl. C12N 15/81; C07K 14/39

(52) U.S. Cl. CPC C12N 15/815; C07K 14/39

(57) ABSTRACT

The invention provides Komagataella phaffii comprising a Per-ARNT-Sim (PAS) gene sequence comprising at least one EcfG binding motif. The PAS gene sequence may encode at least one PAS domain and/or the PAS gene is recombinant or exogenous to Komagataella phaffii. The Komagataella phaffii is preferably able to utilize General Stress Response and/or the Komagataella phaffii preferably has a GSR-inducing signaling pathway.

### TECHNICAL FIELD

The present disclosure provides a method for detecting an outbreak of zoonotic pathogens.

### BACKGROUND OF THE INVENTION

A definition of key concepts is provided, related to this patent invention:

Komagetaella phaffi

Bernauer et al (2020) describes Komagataella phaffii (mistakenly thought to belong to the genus Pichia pastoris) yeast in multiple biotechnology and pharmaceutical industrial applications (Bernauer et al. 2020) as sharing meaningful characteristics from both its ancient ancestors and today's metazoan cells, in eukaryotic molecular cell biology. As a methylotrophic yeast, K. phaffii it is used in research to do with methanol assimilation, peroxisome biogenesis and pexophagy (Bernauer et al. 2020). Yeast genetics research includes mating and sporulation behavior; cellular processes such as protein secretion, lipid biosynthesis and cell wall biogenesis (Bernauer et al. 2020). All strains employed in biotechnology use the established name Pichia pastoris here as a synonym for all Komagataella species, giving reference to the different species for different strains, for specific research experimentation.

Komagataella pastoris (which includes the French strain) and K. phaffii (which includes the American isolates) differ by approximately 10% DNA sequence divergence and two reciprocal translocations (Dalvie et al. 2020), and yet both strains are in use for recombinant protein production under the name P. pastoris. K. phaffii is commercially exploited in the production of biomass and single-cell protein from methanol, including Phillips Petroleum Company (Bartlesville, Okla, United States) and others. Exact strain identifications include CBS2612 (NRRL Y-7556) or CBS7435 (Bernauer et al. 2020).

### Sensors

Sensors describe evidence of driving forces in an ambient environment towards thermodynamic equilibrium, particularly upon the onset of new or increased abiotic and biotic events in the environment. A mechanical sensor may target the physical or chemical attributes of an environmental variation such as temperature, pH, moisture, and so on, while a biosensor might target growthrates, metabolism, soil-nutrient availability, rates of interactions between organisms and expression of resistance to fluctuations in the environment as measures of health and/or disease. And so, the interactions and/or growth is a thermodynamic property, referred to as Gibbs energy in classical and Hamiltonians, in quantum. Ecological sensors directly or indirectly describe methanogenic environments including the ecological carbon cycle and syntrophic interactions of interspecies of H₂, such as the process of H₂ oxidation driven by methyl-reducing methanogenesis and tripartite uptake of formate, electrons and acetate (Nobu et al. 2020). Organism resistance to environmental stresses, such as drought, leads to adaptability in species evolution—also known as fitness. Bacteria use alternative sigma factors binding to RNA polymerase core enzymes in order to respond to environmental stress conditions. Sigma factor EcfG is one of the targets to modify transcription regulation in bacteria and to influence production capacities. To measure organism resistance to environmental stress, sensitivity to proteins and characteristics of a general stress response (GSR) is essential.

### General Stress Response (GSR)

The GSR is a global indicator of different environmental signals via sigma factor-mediated transcription control in bacteria, including most particularly, stress-dependent alternative sigma factors that compete with the housekeeping sigma factor for binding to the RNA polymerase to redirect transcription towards stress response genes when healthy gut microbiome related bacteria become pathogenic due to environment stress affecting the host—e.g., dietary stress, confinement stress, drought stress, and so on. Regulators can either react to the outcome of a stress-related impact on the cellular physiology or by direct sensitivity to the change itself in the ambient environment. There are two families that provide each of these responses: 1. A two-component system which includes a sensor histidine kinase and a cognate response regulator and, 2. alternative sigma factors (as) which are subunits of RNA polymerase holoenzyme that mediate promoter recognition; alternative, non-housekeeping as are widely used in stress responsive signal-transduction pathways. Since every proteobacterial species contain multiple members of each of these families, one of the very first steps in planning design of K. phaffii is to consider if or how it can measure the GSR-related transcriptional response which converts ambient environmental signals into new post-transcriptional proteins within the bacteria and to measure the significance of the stress as high or low based on the degree of those proteins. The ability to sense and relieve the deleterious effects of stress is quick and accurate genetically, and one method involves therefore, the genetic insertion so that K. phaffii will replicate the behavioral response pathways of bacterial organisms seeking stress-survival. Other two-component systems contain Per-ARNT-Sim (PAS) domains which provide feed-forward sensors for signals ranging from variations in light, redox potential, and metabolites to more. And so, while the first step of GSR includes transcriptionally linked genetic regulation of motility and biofilm formation, the rendering of kinases associated with optimization of resources, their allocation and cellular resilience in stressful and changing environments (Gottschlich et al. 2019) makes it simpler and more preferable to use PAS related genes that will respond to the histidine kinases of bacteria, and to thereby report the instant response as an early warning of an environmental stress detection that is always required on behalf of transmembrane autophosphorylation passage, including while additional response regulator proteins will be acting as further transcription factors to change the organism's metabolism, location and more (Chauhan and Calderone 2008). The timeframe for this process can be literally no more than one hour which makes the use of modified-K. phaffii species quite lucrative as an affordable and easily multiplied, distributed habitat-monitoring sensor. Classification of EcfG-regulated genes under low stress conditions with Clusters of Orthologous Groups (COG) categories61 retrieved from the Integrated Microbial Genomes with Microbiome Samples system (IMG/M: https://img.jgi.doe.gov/m/).

### Pathogenic Viruses

Viruses that are part of pandemic infectious outcomes rely on virulence-essential gene products, that do not describe general growth (Chauhan and Calderone 2008). Currently there are no sensors for the gene products that describe virulence from the environment. Climate-related stress events typically result in RNA-virus infection and reinfection of stress-responding hosts (bacteria). Recombination occurs rapidly and largely in order to succeed in reducing dominant hosts a to within that level of nutrient accessibility to all diverse species. Virus activity represents sustainability of all inter-related ecological species that depend on each other at the micro- to macroscale. RNA recombination and reassortment ensure bypass of any host-barriers or resistance via the formation of chimeric molecules from parental genomes of mixed origin, whether within a single genomic segment (“RNA recombination”) or via segmented genomes, as part of an exchange of entire genomic segments between viruses (“reassortment”). One could suggest, virus carry out widespread delivery of changes in order to ensure each and every species has access to the survival of each other, in the greater food-chain, by recombination and reassortment, when a serious or critical environmental stress threatens universal sustainability.

### Genetic Modification

State of the art methods of yeast genetic modification include double strand break repair mechanism, Cre-loxP mediated recombination, Delitto perfetto, Meganuclease mediated double-stranded breaks, or CRISPR/Cas9 system. Gene engineering methods and techniques may require the use of drug-selectable and auxotrophic nutritional markers for validation and maintenance of the new integrated sequence (Fraczek et al. 2018).

### Quaternions

Quaternions (H) are the four-dimensional solution to the multiplication of 3-dimensional vectors involving complex number amplitudes. Multiplication of the amplitudes of two (or more) vectors results in rotation of the vectors, summing their angles in the process.

And so, for a quaternion given by a+bi+cj+dk,

Then,

$${i}^{2} = {j}^{2} = {k}^{2} = ijk = - 1$$

$$i \cdot j = k$$

$$j \cdot k = i$$

$$k \cdot i = j$$

And the reverse of these relationships leads to:

$$j \cdot i = - k$$

$$k \cdot j = - i$$

$$i \cdot k = j$$

These relationships show the non-commutative property of quaternions which removes them from being represented by a Field and that, therefore, separates them from complex numbers.

But, by multiplying quaternions, one can continue working in 3-d space by treating each vector as a dot-product and cross product between them, described in Gibbs-Heaviside equation, i.e.,

$${a}_{1} {v}_{1} = ( {a}_{1} {a}_{2} - {v}_{1} {v}_{2} , {a}_{1} {v}_{2} + {a}_{2} {v}_{1} + ( {v}_{1} \times {v}_{2} ) )$$

it is possible to express any quantum circuit in terms of real gates only.

In quantum computation, gates are matrices that modify the amplitude and rate a phase of each qubit (spinor) in an array of n-registers of qubits. Such are referred to as Pauli matrices. Whole commercial quantum circuit development and simulation resources are available such as by IBM Qiskit, D-Wave, and in government hosted institutions.

### Quaternion Circuits

Fernandez and Schneeberger (2008)(Fernandez and Schneeberger 2003) and Bolokhov (2019) (Bolokhov 2019) and others describe the derivation and correlation of quaternion circuits and quaternionic wavefunctions to quantum circuits and wavefunctions, respectively. A quaterbit is a 2-level system with quaternionic amplitudes. It can be represented by a unit vector |Φ> in a 2-dimensional quaternionic Hilbert space, i.e.

$$\vert Φ 〉 = \hat{α} \vert 0 〉 + \hat{β} \vert 1 〉 ,$$

such that

$${‖ Φ ‖}_{2} = \sqrt{{\vert \hat{α} \vert}^{2} + {\vert \hat{β} \vert}^{2}}$$

up to an arbitrary quaternionic phase factor (Fernandez and Schneeberger 2003). This means:

$$Φ ≡ {Φ}^{′} ⇔ \vert Φ 〉 = \hat{η} \vert {Φ}^{′} 〉 ,$$

$$where$$

$$\vert \hat{η} \vert = 1.$$

Quaternions represent a quater-bit n+1 variation of the quantum qubit. Quaturnion circuits are an array of gates for quaternionic computation using n quaterbits. The quaternion rotation is ideal instead of standard qubit computational gate matrices because of their natural utility for rotation motion based interaction modeling variables. This includes dynamic-moving mobile devices, gaming software algorithms and related (Lanzinger 2020). Quaternions also can be correlated to higher 8-dimensional number systems, octonians (Conway and Smith 2003). The facility for doing so is feasible with radiowaves, transmitted from an arrangement of satellites in a system at Lagrangian-1 between the earth and moon. These radiowaves replace the physical qubit gates by their corresponding electromagnetic frequency modulation resulting in similar information transmission interference and modification.

Complex numbers (Rene Descartes) are the foundation of algebraic geometry in the physical form of the radiowave quantum gates that provide the processing of images, collected from the terrestrial quantum-tactic sensor yeast, such as in human chewing gum or other media where human or other terrestrial species is described as vulnerable socially to a zoonotic disease transmission and infection (Wildberger 2005). For a polynomial P(x, y)=0 the coordinates represent a set of points that satisfy a curve. Linear correspond to degree 1 polynomials. Quadratic polynomials correspond to degree 2 conic sections such as ellipses and hyperbolas. Degree 3 curves (cubic equations) emerge further. Cubic curves are generated by taking a product of linear factors P(x, y)=(a₁, x+b₁, y+c₁)×(a₂x+b₂y+C₂)× . . . ×(aₙx+bₙy+cₙ) which means each of the equations determines a line and then when multiplied together, they produce graph of the intersections of those lines, ie., the polynomial curve is a product of lines. More generally, this type of polynomial is formed initially and then the coefficients are varied which results in algebraic curves (Stillwell 2012). For example, if n=3, the polynomial is a cubic and is a product of three linear factors. Then modify the coefficients to create a continuous curve with additional hyperbolic curves like branches in directions to infinity. The evaluation of these curves by Mobious (homogeneous coordinates) for example of a curve sitting inside a projective plane rather than an affine plane include using 3-dimensional space and looking at a plane going through a point, say z=1. But, instead of x, y as real numbers to describe the polynomial curve, complex numbers satisfy projective geometry and homogeneous coordinates (Moebius and Pluecker, 1830s) for the radiowave-quantum gate properties

The quater-circuit is designed by radiowaves automatically per image according to the quantum-tactic information. Recall that the images are GSR data sensed and transmitted by millions of quantum-tactic yeast on earth. The behaviour of the quater-circuit for each possible gate ordering can be simulated with n+1 qubits. The radiowaves are projected by and received from satellites at Lagrangian-1 between the earth and moon, which is at the ideal quantum-computation temperature and which allows the circuit of radiowave-based quaternium-gates to capitalize on quantum electron storage evolved in natural multidimensional physics not possible on earth, including where such computation involves non-radiowave peripherals in future adaptations to this system. Using this information, simple manipulation of the variables can give results which are easily interpreted, and especially so for the variables which are in quadrature with one another.

Quantum computation (QC) represents bits relevant to quantum states for high-speed quantum information problem-solving, based on superpositions of the states and/or (qu)bit entanglement.

As such, qubit based computation represents information processing through quantum gates, solving (Bounded-error Quantum Polynomial time classes in polynomial time (BQP) (Yamamoto et al. 2015).

Results can be described by the hamiltonian of a complex system of thermodynamic conjugate variables through a quantum circuit simulation which projects particle phase and orientation in the complex space of rotation of data through each qubit.

### SUMMARY OF THE INVENTION

The present disclosure provides a method for using genetic-modified Komagataella phaffii yeast and radiowave photo-imaging satellite arrays at the Earth-Moon Lagragian-1 location. The purpose of the quantum-tactic yeast ingredient and such a longwave optical method is to detect early stress responses in microbiome across a habitat wherever the threat of an outbreak of zoonotic pathogens may emerge suddenly and unnoticeably to any other method of observation currently, and potentially involving zoonotic virus vectors for DNA viruses or epidemic and pandemic related RNA viruses in response to environmental climate stress-conditions that may threaten human populations in the same habitat, thereafter.

The present disclosure particularly provides for Komagataella phaffii comprising at least one (or at least 2, 3, 4, 5, 6, 7, 8, 9, 10) (exogenous/recombinant) nucleotide sequence(s) comprising at least one EcfG binding motif. The nucleotide sequence is preferably a recombinant gene, and/or exogenous to said Komagataella phaffii. Hence, the nucleotide sequence preferably does not naturally occur in said Komagataella phaffii. In addition or alternatively, the nucleotide sequence is a Per-ARNT-Sim (PAS) gene, i.e. encoding at least one PAS domain.

Preferably, the Komagataella phaffii is able to utilize General Stress Response and/or the Komagataella phaffii has a GSR-inducing signaling pathway.

In a preferred embodiment, the EcfG binding motif has (is) a sequence with at least 50, 60, 70, 80, 90, 91, 92, 93, 94, 95, 96, 97, 98, 99, 100% sequence identity with any of SEQ ID NO:1-50, preferably SEQ ID NO:1, as shown in Table 1.

Preferably, the EcfG binding motif in the context of the current invention has a nucleotide sequence with a length of between 15-35 nucleotides, preferably of between 20-30 nucleotides, more preferably of between 23-28 nucleotides.

TABLE 1

| PAS | | |
|---|---|---|
| gene | | Sequence of putative EcfG- |
| ID | Predicted function | binding motif |
| #0072 | ‘CAMP-binding proteins catabolite gene | GGAACGTTACGCCCCCGATCCGTGTT |
| | activator and regulatory subunit of cAMP- | (SEQ ID NO: 1) |
| | dependent protein kinases’ | |
| #0098 | ‘hypothetical protein’ | GGAACAATGCGGGGCCCGTGCGCTTT |
| | | (SEQ ID NO: 2) |
| #0597* | ‘probable methyltransferase’ | GGAACGGCAGGACGGCGTGTGGACGCGCT |
| | | (SEQ ID NO: 3) |
| #0598 | ‘TIGR03440 family protein’ | GCAACTCTTTGAGCTGTCATGCGCT |
| | | (SEQ ID NO: 4) |
| #0628 | ‘Outer membrane protein V’ | GGAAAGATTCCGCAACCGCTGCGTT |
| | | (SEQ ID NO: 5) |
| #0787 | ‘hypothetical protein’ | GGAACGATAGCGGAAACAGGCGGTT |
| | | (SEQ ID NO: 6) |
| #1028 | ‘Uncharacterized protein conserved | GCACCTCAGGCGGCGCCTCCGCCCGTT |
| | in bacteria’ | (SEQ ID NO: 7) |
| #1060 | ‘hypothetical protein’ | GGAACAAGCGGTCGCAGCCTGTGGTT |
| | | (SEQ ID NO: 8) |
| #1102 | ‘Ku protein, prokaryotic’ | GGAACGACTCTTTCTCCCATGAATT |
| | | (SEQ ID NO: 9) |
| #1274* | ‘4-alpha-glucanotransferase’ | CGATCCGCAGGCGCGCGCACGCATT |
| | | (SEQ ID NO: 10) |
| #1275* | malto-oligosyltrehalose trehalohydrolase’ | GGCAGAACCCCGAAGGCCGCGCGCT |
| | | (SEQ ID NO: 11) |
| #1276 | ‘glycogen debranching enzyme GlgX’ | GGGACCTTCCATGCTGCGCCTGCGTT |
| | | (SEQ ID NO: 12) |
| #1281 | ‘Predicted outer membrane protein’ | GGAACGCAGCGCCGCGTCCGAACGTT |
| | | (SEQ ID NO: 13) |
| #1282 | ‘Domain of unknown function (DUF3597).’ | GGAGCATTCGGCGCGCATCGACGTT |
| | | (SEQ ID NO: 14) |
| #1297 | ‘Aerobic-type carbon monoxide dehydrogenase, | GCAACCCGGCGGTGGAGGCCGCTTT |
| | large subunit CoxL/CutL homologs’ | (SEQ ID NO: 15) |
| #1298* | ‘Aerobic-type carbon monoxide dehydrogenase, | GGCACGCCCGACGACCTCCACCCGCT |
| | middle subunit CoxM/CutM homologs’ | (SEQ ID NO: 16) |
| #1299 | ‘Aerobic-type carbon monoxide dehydrogenase, | GGAACGGATGCCAGCAGCATCCCGGT |
| | small subunit CoxS/CutS homologs’ | (SEQ ID NO: 17) |
| #1442 | ‘Predicted small secreted protein’ | GGAACGAATGGCCCGACCGATCATT |
| | | (SEQ ID NO: 18) |
| #1444 | ‘hypothetical protein’ | GGAACCAACGAACGCGACTTTGGTT |
| | | (SEQ ID NO: 19) |
| #1446 | ‘hypothetical protein’ | GGACCCAAAACGGGTTTCGGGGGTT |
| | | (SEQ ID NO: 20) |
| #1462 | ‘Uncharacterized protein conserved in bacteria’ | GCATCCATTTGGAACACGGGTCGTT |
| | | (SEQ ID NO: 21) |
| #1478 | ‘PRC-barrel domain.’ | GGAACCGGCGCGGCCACAAGGCGGTT |
| | | (SEQ ID NO: 22) |
| #1484 | ‘hypothetical protein’ | CGAACTCGTGGCCCGCGTCAGCGCGGT |
| | | (SEQ ID NO: 23) |
| #1611* | ‘Raf kinase inhibitor-like protein, | GCGACGAACGCATCCGCCGCCGATT |
| | YbhB/YbcL family’ | (SEQ ID NO: 24) |
| #1612 | ‘hypothetical protein’ | GCAACCGCCGGGGCCTGTCTTGCGTT |
| | | (SEQ ID NO: 25) |
| #1750 | ‘Glycosyltransferase’ | GGAACAACTCCGCCACGCCGGAGTA |
| | | (SEQ ID NO: 26) |
| #1751 | ‘Glycosidases’ | GGAACCCCCTCGCCCCTCCTCCGTT |
| | | (SEQ ID NO: 27) |
| #1765 | ‘NAD-dependent aldehyde dehydrogenases’ | GGATCGCGTGCCGGGCCTGCGCGTT |
| | | (SEQ ID NO: 28) |
| #1831 | ‘Topoisomerase IB’ | GGAACCGTGCGCGTCGCCTGGGCATT |
| | | (SEQ ID NO: 29) |
| #1836 | ‘Uncharacterized stress protein | GCAACCAACCCCCGATGCGCTCGTT |
| | (general stress protein 26)’ | (SEQ ID NO: 30) |
| #1930 | ‘hypothetical protein’ | GGAACCCTTACCGATGCACGCCGTT |
| | | (SEQ ID NO: 31) |
| #2026 | ‘hypothetical protein’ | GGAACTTCGGTCCAAATGGTGCGTT |
| | | (SEQ ID NO: 32) |
| #2180 | ‘hypothetical protein’ | GGAACCGCCGAGCGGATCGCGCGTT |
| | | (SEQ ID NO: 33) |
| #2452 | ‘hypothetical protein’ | GATACGCCCGCGATCCCAACGCGTT |
| | | (SEQ ID NO: 34) |
| #2489 | ‘Catalase.’ | CGATCGGCTGCGGCATCTGCGCATT |
| | | (SEQ ID NO: 35) |
| #2499 | ‘Zn-dependent alcohol dehydrogenases’ | GGCACGGCGCTGTGCGACGCCGGCT |
| | | (SEQ ID NO: 36) |
| #2509 | ‘hypothetical protein’ | GAAACGCCCGCCCAGCCCAGCCAGCCGGT |
| | | (SEQ ID NO: 37) |
| #2510 | ‘hypothetical protein’ | GCAACCCGCTGGGCTGCGGGACGTT |
| | | (SEQ ID NO: 38) |
| #2527 | ‘Outer membrane protein’ | GGATCAACCGCGGGGGACGGCCGTT |
| | | (SEQ ID NO: 39) |
| #2528* | ‘Uncharacterized protein conserved in bacteria’ | GCGACGCGCTGGCGCCATCGCGCCGCT |
| | | (SEQ ID NO: 40) |
| #2534 | ‘hypothetical protein’ | GGAACGGCTCCATTCCTGACCAGTT |
| | | (SEQ ID NO: 41) |
| #3063 | ‘Protein required for attachment to host cells’ | GCAACCCGCCCGCCTCGGCTGCGTT |
| | | (SEQ ID NO: 42) |
| #3142 | ‘hypothetical protein’ | GGAACCGGTATCGCCTGCGTAACGTT |
| | | (SEQ ID NO: 43) |
| #3266 | ‘Uncharacterized conserved protein’ | GGTGCGCCGGCGTGCGCTCGCCGCT |
| | | (SEQ ID NO: 44) |
| #3278 | ‘hypothetical protein’ | CGAACCGTTTCCCCCGTCCGCTCGTT |
| | | (SEQ ID NO: 45) |
| #3404 | ‘Predicted integral membrane protein’ | GGAACAGATGCTGGCCCAGCGCCTCGTC |
| | | (SEQ ID NO: 46) |
| #3506* | ‘Predicted glycosyl transferase’ | GCCACGCCCCGGCCTGCTGCGCGTG |
| | | (SEQ ID NO: 47) |
| #3507* | ‘Predicted glycosyltransferases’ | GGCTCGAAGCGCTCGCCGACCGGTT |
| | | (SEQ ID NO: 48) |
| #3508* | ‘Glycosyltransferase’ | GAAACCTTCCTGCGCGTCGGCGGCT |
| | | (SEQ ID NO: 49) |
| #3509* | ‘Exopolysaccharide biosynthesis protein’ | GGTCGTCGGCGACGCCGGCCGGTT |
| | | (SEQ ID NO: 50) |

In an embodiment, the K. phaffii disclosed herein measures the significance of the stress (e.g. as high or low based or absent) on the degree of General Stress Response, which can be detected for example by measuring the transcription (mRNA levels) of the putative EcfG-in the context of the current invention. In addition or alternatively, the amount of transcribed proteins can be measured, such as by UV absorbance.

State of the art methods of yeast genetic modification can be used to obtain said K. phaffii, including double strand break repair mechanism, Cre-loxP mediated recombination, Delitto perfetto, Meganuclease mediated double-stranded breaks, or CRISPR/Cas9 system.

In an embodiment, the PAS domain sequence as disclosed herein is introduced into K. Phaffi using a nucleic acid delivery construct comprising the PAS gene sequence preferably at least one sequence encoding an EcfG binding motif. The nucleic acid delivery construct in the context of the current invention is preferably chosen from one or more of a plasmid, a recombinant adenovirus, an adeno-associated virus (AAV), a retrovirus, a lentivirus, a herpes simplex virus, and a vaccinia virus, preferably a lentivirus.

It is found that Komagataella phaffii comprising a recombinant Per-ARNT-Sim (PAS) gene sequence comprising at least one EcfG binding motif can detect bacterial stress conditions in methanogenic environments. These findings could be established in in vitro laboratory settings, however the implications of the current invention go beyond those that can be readily verified in a laboratory environment. For example, the current invention is particularly effective when used in combination with advanced detection techniques that are target-specific for pathogen evolution and compatible with a use in necessary habitats to confer the pathogen ecology, including their changes in nucleic acids, proteins, virions, and viability (and which cannot be replicated in a laboratory setting). Such conditions are more challenging or even impossible to determine in a laboratory setting. For example, herein a longwave optical method is presented which can be used in the context of the current invention and which can detect the wavelength of proteins associated with the RNA transcription regulation that follows from the PAS gene expression upon stress variable occurrence. The applicability of the longwave optimal methods are also considered to go beyond the detection methods suitable for verification in a laboratory setting. The current invention may also be used to show induction or blockage of effector genes required for natural genetic transformation of pathogens in a given habitat. Such genes appear to remain nontransformable under non-natural conditions and therefore are hard to study under laboratory conditions.

The range of wavelengths of the detection method as taught herein may correspond to UVC wavelengths represented by UVC photon dissipation that would take place in the period of nucleotide codons (or anticodons) for each selected gene.

Also provided are detection means to detect the wavelength of proteins associated with the RNA transcription regulation that follows from the PAS gene expression upon stress variable occurrence. The range of wavelengths may correspond to UVC wavelengths represented by UVC photon dissipation that would take place in the period of nucleotide codons (or anticodons) for each selected gene.

In an embodiment, the EcfG binding motif allows determining the level of stress and/or the General Stress Response.

In an embodiment, the transcription of the sequence encoding an EcfG binding motif allows determining the level of stress and/or the General Stress Response.

A preferred embodiment is to distribute and/or grow the yeast over (extended) areas and/or multiple locations on earth's surface.

The term ‘sequence identity’ or ‘sequence similarity’ as used herein refer to a situation where an amino acid or a nucleic acid sequence has sequence identity or sequence similarity with another reference amino acid or nucleic acid sequence. ‘Sequence identity’ or ‘sequence similarity’ can be determined by alignment of two polypeptides or two nucleotide sequences using global or local alignment algorithms. Sequences may then be referred to as “substantially identical” or “essentially similar” when they (when optimally aligned by for example the programs GAP or BESTFIT using default parameters) share at least a certain minimal percentage of sequence identity (as defined below). GAP uses the Needleman and Wunsch global alignment algorithm to align two sequences over their entire length, maximizing the number of matches and minimises the number of gaps. Generally, the GAP default parameters are used, with a gap creation penalty=50 (nucleotides)/8 (proteins) and gap extension penalty=3 (nucleotides)/2 (proteins). For nucleotides the default scoring matrix used is nwsgapdna and for proteins the default scoring matrix is Blosum62 (Henikoff & Henikoff, 1992, PNAS 89, 915-919). Sequence alignments and scores for percentage sequence identity may be determined using computer programs, such as the GCG Wisconsin Package, Version 10.3, available from Accelrys Inc., 9685 Scranton Road, San Diego, CA 92121-3752 USA, or EmbossWin version 2.10.0 (using the program “needle”). Alternatively percent similarity or identity may be determined by searching against databases, using algorithms such as FASTA, BLAST, etc. Preferably, the sequence identity refers to the sequence identity over the entire length of the sequence.

In this patent, the goal of genetic modification is to enable K. phaffii as an intermediate sensor-transmitter of GSR-activating stress signals to subsequent receiving image interpreters, the radiowave-driven quatercircuits between satellites at Lagrangian-1 of the Earth-Moon orbit. As mentioned above, early detection of GSR is feasible by direct synchronisation of the K. phaffii with the bacteria stress signal activation within the hour of GSR activation using PAS genes inserted in the K. phaffii. These genetically-modified organisms will join EcfG-dependent genes involved in the GSR-functions: PhyR, NepR and PhyT (Kaczmarczyk et al. 2011) (Gottschlich et al. 2018) as well as activity from additional regulators that include the important orphan signal-sensing hybrid histidine kinase (#1746) under low stress conditions and via sigma factor competition (Gottschlich et al. 2019). It is worth noting that this includes a powerful opportunity to encode the protein that is annotated as a membrane-bound Fe2+-dicitrate sensor (#1210) within the ‘FecR superfamily’ (NCBI Conserved Domain Search) (Gottschlich et al. 2019) so that a RNA polymerase sigma factor (sigma-70 family) (#1209) and a protein annotated as an outer membrane receptor for ferrienterochelin and colicins (#1208) (Gottschlich et al. 2019). These genes appear to play a role in direct regulation of iron homeostasis and to counteract the production of reactive oxygen species (ROS) during an activated GSR in the wild (according to the published research) which may be useful if the modified-K. Phaffii ingredient is to be used as a vehicle for any other purpose such as the evaluation of competition with other species in the shared microbiome environment, rather than remote-imaging and to recognise inter-species communication per microbiome, e.g., that would reveal additional information such as the signaling cascade activation and gene expression encountered in species self-defense (Sharifi and Ryu 2021) generally.

In this patent, a method for quaternion circuit is envisioned using any state-of-the-art qubit based circuit algorithm of traditional physical systems except that, radiowaves are deployed rather than physical models. Furthermore, the quantum image analysis is envisioned to be computed by radiowave-gates delivered by radiowave-transmitting satellites in an array together, located at Lagrangian-1 (L-1) of the earth and moon. This location provides ideal and essential deep temperature and freedom from obstruction-error management found with high-cost resources of a terrestrial quantum simulation computer-counterpart provided commercially for small qubit-ranges such as by IBM, Google, and others. Lagrangian points are where all the gravitational forces acting between two objects cancel each other out and therefore can be used by spacecraft to ‘hover’. L-1 affords the opportunity to deliver unlimited quantum image processing of images including the opportunity to capitalize on quantum information storage in multiple dimensions for analysis of large-scale GSR response dimensions.

In radiowaves, where the wavelength ranges from 1 m (T.V. or radiowave) to more than 10⁵ m (longwave radio), the frequency rate is 10^{8.5} Hz to more than 10³ kilocycles respectively, the information is converted from millions to trillions of ecohabitat-wide PAS-modified K. phaffii signaling as an image by a radiowave satellite. In general, the electromagnetic carrier wave operates as amplitude modulation (AM) or as frequency modulation (FM) or in digital form (pulse modulation). Frequency band width is proportional to the information density above 10,000 Hz.

In this patent, the radiowave-emitting and receiving satellite array are therefore suitable to host an algorithm that can simulate a circuit composed of quantum gates that manage the superposition and entanglement of qubits, no less efficiently and precisely than in quantum optics. In fact, the relationship between current state-of-the-art satellites that provide radiowave width control apertures, facilitates the study of different multidimensional layers of stress-response images such as for multiple PAS-modified K. phaffii stress-type sensors, each according to a specific feature of the environment of the habitat for which the monitoring is required. In this way, the basis for examining the source and arrival of virulent-pathogens emergence based on conditions in the habitat can be observed at a fine-grain volume of detail in the very hour that the GSR response initiates. By comparison, the sort of analysis is severely restricted or impossible to generate globally and continuously in a laboratory environment.

### Lagrangian 1

This patent envisions the creation of qubit-gates in an assembly that involves radiowave-driven quantum-gates at a location where gravitational forces are zero on any qubits and temperature is 2.7 kelvins which is ideal for quatercircuits. The distance between the Moon and the Lagrangian point L1 equals 61350 km approximately; the Earth-to-L1 distance equals 323050 km. There are additional Lagrangian points for which low-energy radiowave transmission research exists.

Lunar Radiowaves into Quaternion Gates

In traditional quantum computers, semiconductor structures such as nanosized quantum dots can be scaled into transistor-like equivalents of a standard computer chip in order to provide n-qubit gates in corresponding quantum circuits (Hendrickx et al. 2021).

The principles of the quantum circuit are the same, meaning that the end result of the circuit ensures that the state vectors always have real amplitudes, but unitary transformations (on the complex Hilbert space) representing the gates are allowed, as long as the end result is still a real amplitude vector. This ensures the completeness with classical signal evaluation which must be done in order to verify and optimise the radiowave circuit algorithm output which is driven by the genetically-modified K. phaffii signal input.

Lunar quatercircuit-building Radiowaves requires deployment of any state-of-the-art space-based antenna array comprising of numerous satellites, capable of synthesizing a specified range of radiowavelengths (apertures).

The PAS can be any genomic insertion relevant to a specific environment-stress parameter of interest, including light, salt, oxidation-stress, acid, etc. In general, an array of satellites emits as well as reads radiowaves that carry optical photons. Radioreflectors focus the radiowaves on specific antenna in order so that the amount of detail obtained from an image from each PAS-modified K. phaffii organism (ordinarily limited by the wavelength of the photons divided by the size of the aperture), is detectible by optical photons, several hundred nanometers in size, with wavelengths up to thousands times longer than light. In addition, this patent describes that the image or map “viewed” by the array of satellites can facilitate the descrambling of the image according to gradients of stress by using the quantum-circuit simulation of each PAS-modified K. phaffii ingredient as the equivalent of one qubit. The orientation of satellite axes means that the quantum circuit is described as quatercircuit simulations in the algorithm, based on quaterimages of the habitat and quaterbits, the PAS-modified K. phaffii species. The output of the radio wave-simulation of the quantum-circuit as a quatercircuit supports the “close-up” of microbiomes within hosts per habitat, rather than simply mapping a 2D-spread in traditional mapping images of landscapes and oceans.

Currently, at the time of this patent-writing, space-based aperture array for astronomical observations in the Ultra-Long Wavelength (ULW) regime of greater than 10 m lies deliver radiowave below 30 MHz, and 1 MHz limited band (with 1 kHz channels) accommodates the detection of one million astronomical sources in a five year duration mission. (Rajan et al. 2016).

The quantum image of such PAS-modified K. phaffii can use any image-processing quantum circuit.

The qubit is the element of information carriage.

The state of a qubit is defined by its 2-dimensional state vector, namely:

$$\vert Φ >= α \vert 0 > + β \vert 1 > ,$$

$$such that$$

$${‖ Φ ‖}_{2} = \sqrt{{\vert α \vert}^{2} + {\vert β \vert}^{2}} = 1 ,$$

Where

|0> and |1> are the two canonical basis vectors
and

$$Φ ≡ {Φ}^{′} ⇐ ⇒ \vert Φ >= {e}^{i Θ \vert} \vert {Φ}^{′} >$$

$$where$$

$$Θ ϵ [ 0 , 2 π >$$

### Details

In this patent, gradients of GSR are measured by K. phaffii sensors that are used as ingredients in ecologically-relevant media wherever their growth from methanol carbon sources can be supported in a given habitat.

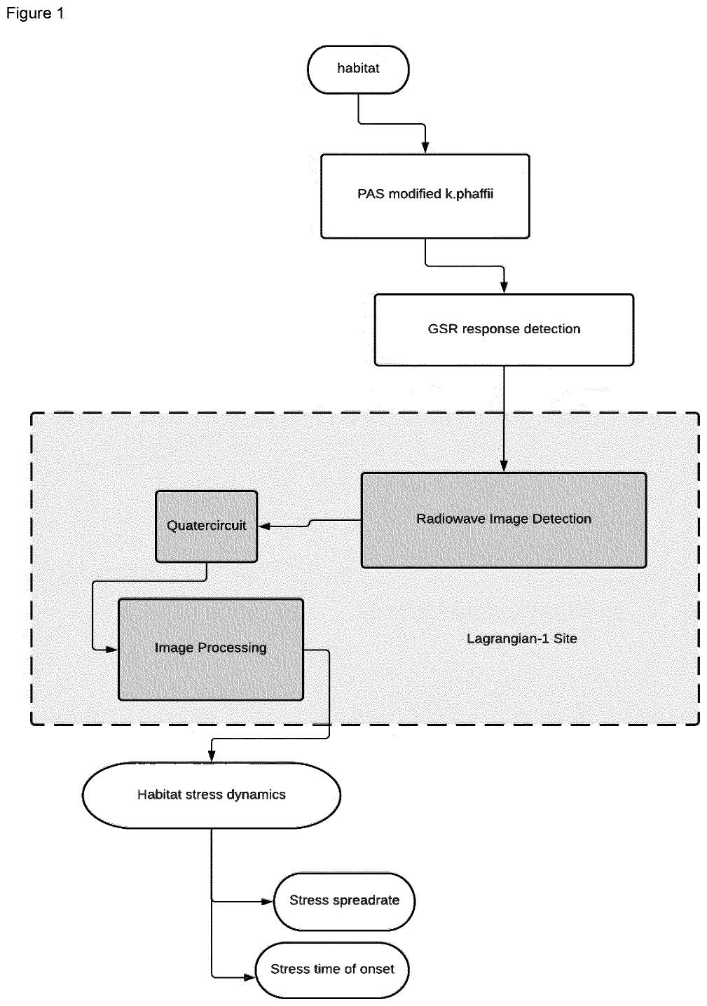

FIG. 1 shows a schematic of the process.

The habitat may represent one in which human safety is suspected to harbour risk of disease-spread or other widespread threat, both natural and manmade. The basis for PAS-modified K. phaffii is that it is a K. phaffii genome that has been modified with any EcfG gene combination that supports one or multiple PAS environmental-sensor readiness, where the environmental stress my include oxidative-stress, acid-stress, salt-stress, light-stress, drought-stress or any kind of combination of gene-related PAS module associated with stress-responses across all kingdoms of species that may be inserted into the genome by standard genetic-modification methods on behalf of ambient pathogen behavior initiation and subsequent pathway monitoring (spread) from a source to a target across one or more habitats. The use of these PAS-modified K. phaffii sensors delivers an image to a corresponding radiowave satellite of array of satellites that are located on Lagrangian-1 of the Earth-Moon orbit. Therefore the radiowaves are sensitive to the PAS-modified K. phaffii sensor reaction to the ambient environment stress and can use radiowaves in the scan of the habitat for these sensors at sufficiently long wavelengths in order to finetune the optical photonic scale in which to render an image of the entire habitat based on low, medium or high-stress in the microbiome of diverse bacterial species. Accordingly, the method of designing radiowaves is already in existence in previous patents not written by the inventor but which may be considered current state of the art and applicable to develop the optic scale. However, the nature of sensor proteins which includes a set of common genetic signals during GSR stress is on behalf of those that represent the motility and generation of biofilm which are inherently the mechanisms of spread of proteobacteria that become host to pathogenic virus and human infection spread and human infection manifestation for such viruses. And so, rather than the classical system of virus evaluation after an infection has already manifested such spread behavior for the features of motility or other kinds of GSR response, the satellite-array at Lagrangian-1 is assembled to receive the image from the PAS-modified K. phaffii as an n-register of quaterbits so that the microbiotic species stress signals can be analysed in an image based on quaternion gates in a corresponding circuit (quatercircuit). This circuit is no different from a quantum circuit except that the transformation is non-commutative and requires registration of 2-bit addition to each gate. In this way, the image can be analysed for the direction of spread of changes due to stress at the bacterial level across diverse species using only three gray-scale colours instantly and with full sensitivity in the same hour that an average GSR-response lasts, and which is typically impossible to recognise by any other classical habitat-monitoring system. The collection of these nanosized signals is therefore rendered with geo timestamps in hourly durations.

### EMBODIMENTS

1. A method by which to modify K. phaffii yeast for environmental stress response mapping:
   a. Select a single or shared environmental bacterial-stress PAS-variable and the corresponding transcription factor (PAS)
   b. Use the BacTFDB database to select corresponding EcfG gene
   c. Insert the gene(s) into a Komagataella phaffii organism with the most suitable genomic modification technique double strand break repair mechanism, Cre-loxP mediated recombination, Delitto perfetto, Meganuclease mediated double-stranded breaks, or CRISPR/Cas9 system.
   d. Ensure at least a 50% distribution of this newly-developed K. phaffii mutant so that it expresses its PAS-modified EcfG module in a self-sustaining habitat-wide growth rate; this means it requires access to methanol or corresponding carbon-source and to be a living ecosensor the same microbiome environment.
2. Configure a quatercircuit-generating algorithm for stress-response and related pathogen spread reporting at the bacterial level of a habitat
   a. Replace any standard quantum n-register qubit based circuit for quantum image generation with an n+1-register qubit based circuit and the following rules:
      i. Initialise the circuit
      ii. Simulate the circuit. An example from Fernandez and Schneeberger (2003) (Fernandez and Schneeberger 2003):
         1. For a generic n+1 qubit quantum circuit with operator U, composed of s elementary gates, order the gates and serialize the circuit. For example, U_{c}=U^{(s)} U^{(s-1)} . . . U⁽²⁾ U⁽¹⁾
         2. For each gate gϵ{1, . . . , s}replace the n-ary operation U^{(g)} corresponding to the g-th gate, with an adequate real circuit O^{(g)} simulating it.
         3. Construct the overall real circuit C^{(g)} by concatenating the circuits for each level gin the same order as in Step 1, i.e, if O_{c} is the operator for C^{(g)} then let O_{c}=O^{(g)} . . . O⁽²⁾ O⁽¹⁾.
         a. the images to be used to initialise the circuit C^{(g)} should be defined as simply |Ψ₀> and |Ψ₁>
         4. Write a description of the real circuit C^{(g)} and of its input state and ask the real computing ‘oracle’ to provide the result of a measurement on its final state.
         5. Perform the classical post-processing on the result of the measurement and provide the classical answer.
   b. For the management of the circuit across unknown variable habitat size:
      i. width is always increased by only one, but circuit depth can be equal to the circuit size in the worst case
      ii. the original circuit given is constructed with some set of universal gates, formed by modified radiowaves electromagnetically in either a dedicated satellite or jointly between the satellites which collect and prepare the image from earth. If so, then the circuit simulation by these satellites or that satellite will depend on d, the number of quaterbits streamed by the K. phaffii, which become known henceforth as quantum-tactic or quater-tactic yeast ingredients of the quantum image in the largest gate, in the universal set, in the circuit. In particular, if d>3 that the gate can be decomposed into Q_{g} a set of elementary 3-, 2- or 1-qubit gates, universal for quantum computing, without loss of generality when it is as simple as a 2d×2d quaternion matrix.
      iii. the image quantum operator U_{g}=ĥ(Q_{g}) may also be included in the set of elementary gates. U_{g} is now a 2(d+1)×2(d+1) matrix.
      iv. Any temporal chain σ of an n-quaterbit quaternionic circuit can be exactly simulated by an (n+2)-real (classical) bit circuit
      v. The phase information is defined by a unit quaternion, derived from the quantum-tactic yeast which cannot be represented by just one radiowave image formed at an angle of radiowave projection as is the case that might be done mathematically for a unit complex number.
      vi. the output of a quaternionic circuits depends on the order of evaluation of the gate quaternionic amplitude, i.e., the “evaluation path” of the circuit for investigating the image, as there is no unique circuit operator for all possible ways of re-combining the gates.
   c. Use ultralow frequency distributed aperture array radiowave optics from satellite necessarily at Lagrangian-1 such that orientation is towards the pulse-like expression of the genetically-expressed PAS-modified K. phaffii signal proteins expressed
      i. Report images according to patterns of one-hour maximum durations of these proteins including ferric related oxidative reactive stress encountered, bacterial motility, changes in metabolism and DNA-repair to such degree that the radio wavelength frequency from the satellite matches the wavelength of these proteins each of which is RNA-transcription regulated and therefore minimized in pulses, within the cellular organism throughout the habitat.
   d. Prior to evaluation, calibrate the PAS-modified K. phaffii stress-signaling into corresponding radiowave apertures for which the photonic optical time lens is versatile and sufficient to capture the controlled interplay between the stress-proteins, dispersion of bacteria (motility) and phase-modulation in multi-radiowave satellite scanning; it is important that the image-capture be phase-preserving so that the progression of stress leading to the death of organisms is also coherently monitored for all types of stress signals that the bacteria may have for its relocation (motility) and survival (repair) and multiplication (biofilm adhesion and growth) in a new habitat (pathogenic or non-pathogenic).
   e. Establish quantum-image processing
      i. The simulation algorithm follows any textbook description for an n-qubit quantum circuit with operator U_{c}, composed of n elementary gates.
      ii. Include the limiting factors of inter-satellite link bandwidths in the image processing.
      iii. Include interference levels from the Earth, based on the number of sampling quaterbits, relative speed-vectors of the satellite nodes, and orbit quater-image maintenance, and achievable down-link bandwidth to Earth.
      iv. Stream radiowaves in coherent multi-level modulation formats at the same range of wavelength for the EcfG gene expression responsible for reporting a PAS-modified K. phaffii stress response.
      v. Continue to use phase-modulated radiowaves to deliver the essential equivalent of a remote environmental microscope capable of genetic details, including where the details are processed by a quater-circuit yielding a quater-image that forecasts the scale of environmental threat to bacteria growth, volume of bacteria death or evidence of interim dying of bacteria across the habitat in sufficient map colourization such as light-grey, growth; grey, interim progression or dying; black, death or species wipe-out.
   f. Compare with regional biothermodynamics of the habitat

### REFERENCES

Bernauer, Lukas, Astrid Radkohl, Leonie Gabriela Katharina Lehmayer, and Anita Emmerstorfer-Augustin. 2020. “Komagataella phaffii as Emerging Model Organism in Fundamental Research.” Frontiers in Microbiology 11: 607028.
Bolokhov, Pavel A. 2019. “Quaternionic Wave Function.” International Journal of Modern Physics. A, Particles and Fields, Gravitation, Cosmology 34 (02): 1950001.
Chauhan, Neeraj, and Richard Calderone. 2008. “Two-Component Signal Transduction Proteins as Potential Drug Targets in Medically Important Fungi.” Infection and Immunity 76 (11): 4795-4803.
Conway, John H., and Derek A. Smith. 2003. On Quaternions and Octonions. CRC Press.
Dalvie, Neil C., Justin Leal, Charles A. Whittaker, Yuchen Yang, Joseph R. Brady, Kerry R. Love, and J. Christopher Love. 2020. “Host-Informed Expression of CRISPR Guide RNA for Genomic Engineering in Komagataella phaffii.” ACS Synthetic Biology 9 (1): 26-35.
Fernandez, Jose M., and William A. Schneeberger. 2003. “Quaternionic Computing.” arXiv [quant-Ph]. arXiv. http://arxiv.org/abs/quant-ph/0307017.
Fraczek, Marcin G., Samina Naseeb, and Daniela Delneri. 2018. “History of Genome Editing in Yeast.” Yeast 35 (5): 361-68.
Gottschlich, Lisa, Miriam Bortfeld-Miller, Christoph Gabelein, Sebastian Dintner, and Julia A. Vorholt. 2018. “Phosphorelay through the Bifunctional Phosphotransferase PhyT Controls the General Stress Response in an Alphaproteobacterium.” PLoS Genetics 14 (4): e1007294.
Gottschlich, Lisa, Petra Geiser, Miriam Bortfeld-Miller, Christopher M. Field, and Julia A. Vorholt. 2019. “Complex General Stress Response Regulation in Sphingomonas Melonis Fr1 Revealed by Transcriptional Analyses.” Scientific Reports 9 (1): 1-13.
Hendrickx, Nico W., William I. L. Lawrie, Maximilian Russ, Floor van Riggelen, Sander L. de Snoo, Raymond N. Schouten, Amir Sammak, Giordano Scappucci, and Menno Veldhorst. 2021. “A Four-Qubit Germanium Quantum Processor.” Nature. https://doi.org/10.1038/s41586-021-03332-6.
Kaczmarczyk, A., S. Campagne, F. Danza, L. C. Metzger, J. A. Vorholt, and A. Francez-Charlot. 2011. “Role of Sphingomonas Sp. Strain Fr1 PhyR-NepR-EcfG Cascade in General Stress Response and Identification of a Negative Regulator of PhyR.” Journal of Bacteriology. https://doi.org/10.1128/jb.06006-11.
Lanzinger, Franz. 2020. “Programming C# in Unity.” 2D Game Development with Unity. https://doi.org/10.1201/9780429328664-2.
Nobu, Masaru K., Takashi Narihiro, Ran Mei, Yoichi Kamagata, Patrick K. H. Lee, Po-Heng Lee, Michael J. McInerney, and Wen-Tso Liu. 2020. “Catabolism and Interactions of Uncultured Organisms Shaped by Eco-Thermodynamics in Methanogenic Bioprocesses.” Microbiome 8 (1): 1-16.
Rajan, Raj Thilak, Albert-Jan Boonstra, Mark Bentum, Marc Klein-Wolt, Frederik Belien, Michel Arts, Noah Saks, and Alle-Jan van der Veen. 2016. “Space-Based Aperture Array for Ultra-Long Wavelength Radio Astronomy.” Experimental Astronomy 41 (1): 271-306.
Sharifi, Rouhallah, and Choong-Min Ryu. 2021. “Social Networking in Crop Plants: Wired and Wireless Cross-Plant Communications.” Plant, Cell & Environment 44 (4): 1095-1110.
Stillwell, John. 2012. Mathematics and Its History. Springer New York.
Wildberger, Norman John. 2005. Divine Proportions: Rational Trigonometry to Universal Geometry. Wild Egg Sydney.
Yamamoto, Satoru, Shigeaki Nakazawa, Kenji Sugisaki, Kazunobu Sato, Kazuo Toyota, Daisuke Shiomi, and Takeji Takui. 2015. “Adiabatic Quantum Computing with Spin Qubits Hosted by Molecules.” Physical Chemistry Chemical Physics: PCCP 17 (4): 2742-49.

### EXAMPLE

### Example 1

The Per-Arnt-Sim (PAS) domain is a molecular sensor for environmental conditions such as temperature, humidity, and pH. The alternative sigma factor EcfG induces General Stress Response (GSR) by modifying transcription regulation by binding to RNA polymerase. It is investigated if Komagataella phaffii (K. Phaffi) and other yeasts comprising a recombinant Per-ARNT-Sim (PAS) gene sequence comprising at least one EcfG binding motif can measure the significance of the stress based on the degree of General Stress Response (GSR).

A plasmid comprising a PAS domain, with or without (controls) putative EcfG-binding sequence is introduced into K. phaffii. The following EcfG-binding sequences are tested:

(SEQ ID NO: 1)
GGAACGTTACGCCCCCGATCCGTGTT
(SEQ ID NO: 5)
GGAAAGATTCCGCAACCGCTGCGTT
(SEQ ID NO: 10)
CGATCCGCAGGCGCGCGCACGCATT
(SEQ ID NO: 26)
GGAACAACTCCGCCACGCCGGAGTA
(SEQ ID NO: 29)
GGAACCGTGCGCGTCGCCTGGGCATT

A low stress condition is established by exposing K. phaffii to hydrogen peroxide (H₂O₂)(Lin et al. Biotechnol Biofuels. 2021 Jul. 20; 14(1):160)). A high stress condition is established by exposing K. phaffii in combination with increased temperature (Zhong et al. Microb Cell Fact. 2014 Nov. 26; 13:163).

Table 2 shows the relative mRNA levels for the respective gene transcripts of the EcfG-binding sequences. This method establishes whether there is a correlation between the level of GSR and the gene transcript.

It is shown that the generated K. phaffii having PAS domain comprising an EcfG-binding binding sequence can measure the significance of the stress level induced. The significance of the stress level is most accurately established with K. phaffii having PAS domain comprising sequence GGAACGTTACGCCCCCGATCCGTGTT (i.e. SEQ ID NO:1, Group 3 in Table 2). For the control with the same PAS domain but without EcfG binding motif (Group 1 in Table 2), no or very low level of transcription occurs of sequences according to SEQ ID NO:1, 5, 10, 26, and 29 in all levels of stress. Similarly, K. phaffii generated to express aryl hydrocarbon receptor containing PAS domain (as according to Zheng et al. Protein Expr Purif. 2016 June; 122:72-81, Vazquez-Rivera et al. Toxicol Rep. 2021 Nov. 26; 9:1-11), no or very low level of transcription occurs of sequences according to SEQ ID NO:1, 5, 10, 26, and 29 in all levels of stress (Group 2 in Table 2).

It is anticipated that similar results are obtained with the other sequences listed in Table 1, as they all share the common feature of being putative EcfG-binding motifs.

Table 3 shows the comparison of K. phaffi, Saccharomyces cerevisiae (S. cerevisiae), Sphingomonas melonis (S. melonis) having PAS domain comprising sequence GGAACGTTACGCCCCCGATCCGTGTT (i.e. SEQ ID NO:1), in terms of establishing a possible correlation between the level of GSR and the gene transcript. It appears that the significance of the stress level is most accurately established with K. phaffii, in particular compared to non-methylotrophic yeasts.

Without being bound by theory, the transcription level of the putative EcfG binding motifs appear to correlate with the level of the stress conditions, and the EcfG binding motifs may therefore be involved in fine-tuning the GSR. The transcriptional profile appears to be most congruent with the stress condition in K. phaffi, which may be most sensitive in detecting stressful conditions and propagation of the GSR-related pathways, in particular in methanogenic conditions.

TABLE 2

| | Putative EcfG-binding | Stress condition | | |
|---|---|---|---|---|
| Group | sequence | No stress | Low stress | High stress |
| 1 | Control 1 | Absent/very | Absent/very | Absent/very |
| | PAS without EcfG binding | low | low | low |
| | motif | | | |
| 2 | Control 2 | Absent/very | Absent/very | Absent/very |
| | PAS comprised in AHR | low | low | low |
| | Without EcfG binding motif | | | |
| 3 | GGAACGTTACGCCCCCGATCCGTGTT | Absent | Low/Average | Very high |
| | (SEQ ID NO: 1) | | | |
| 4 | GGAAAGATTCCGCAACCGCTGCGTT | Absent/very | Low/Average | High/Very |
| | (SEQ ID NO: 5) | low | | high |
| 5 | CGATCCGCAGGCGCGCGCACGCATT | Absent/very | Low/Average | High/Very |
| | (SEQ ID NO: 10) | low | | high |
| 6 | GGAACAACTCCGCCACGCCGGAGTA | Absent/very | Low/Average | High/Very |
| | (SEQ ID NO: 26) | low | | high |
| 7 | GGAACCGTGCGCGTCGCCTGGGCATT | Absent/very | Low/Average | High/Very |
| | (SEQ ID NO: 29) | low | | high |

TABLE 3

Stress condition

| Yeast | No stress | Low stress | High stress |
|---|---|---|---|
| K. phaffi | Absent | Low/Average | Very high |
| S. cerevisiae | Absent | Absent/very low | Low/average |
| S. melonis | Absent | Absent/very low | Low/average |

1. Komagataella phaffii comprising a Per-ARNT-Sim (PAS) gene sequence comprising at least one sequence encoding an EcfG binding motif, wherein the sequence encoding the EcfG binding motif has at least 90% sequence identity with any one of SEQ ID NO:1-50.

2. Komagataella phaffii according to claim 1, wherein the PAS gene sequence encodes at least one PAS domain and/or wherein the PAS gene is recombinant or exogenous to Komagataella phaffii.

3. Komagataella phaffii according to any one of the previous claims, wherein the Komagataella phaffii is able to utilize General Stress Response (GSR) and/or wherein the Komagataella phaffii has a GSR-inducing signaling pathway.


SEQUENCE LISTING (50 sequences)

SEQ ID NO: 1 - DNA, 26 bp, Artificial Sequence
  Sphingomonas melonis Fr1
ggaacgttacgcccccgatccgtgtt

SEQ ID NO: 2 - DNA, 26 bp, Artificial Sequence
  Sphingomonas melonis Fr1
ggaacaatgcggggcccgtgcgcttt

SEQ ID NO: 3 - DNA, 29 bp, Artificial Sequence
  Sphingomonas melonis Fr1
ggaacggcaggacggcgtgtggacgcgct

SEQ ID NO: 4 - DNA, 25 bp, Artificial Sequence
  Sphingomonas melonis Fr1
gcaactctttgagctgtcatgcgct

SEQ ID NO: 5 - DNA, 25 bp, Artificial Sequence
  Sphingomonas melonis Fr1
ggaaagattccgcaaccgctgcgtt

SEQ ID NO: 6 - DNA, 25 bp, Artificial Sequence
  Sphingomonas melonis Fr1
ggaacgatagcggaaacaggcggtt

SEQ ID NO: 7 - DNA, 27 bp, Artificial Sequence
  Sphingomonas melonis Fr1
gcacctcaggcggcgcctccgcccgtt

SEQ ID NO: 8 - DNA, 26 bp, Artificial Sequence
  Sphingomonas melonis Fr1
ggaacaagcggtcgcagcctgtggtt

SEQ ID NO: 9 - DNA, 25 bp, Artificial Sequence
  Sphingomonas melonis Fr1
ggaacgactctttctcccatgaatt

SEQ ID NO: 10 - DNA, 25 bp, Artificial Sequence
  Sphingomonas melonis Fr1
cgatccgcaggcgcgcgcacgcatt

SEQ ID NO: 11 - DNA, 25 bp, Artificial Sequence
  Sphingomonas melonis Fr1
ggcagaaccccgaaggccgcgcgct

SEQ ID NO: 12 - DNA, 26 bp, Artificial Sequence
  Sphingomonas melonis Fr1
gggaccttccatgctgcgcctgcgtt

SEQ ID NO: 13 - DNA, 26 bp, Artificial Sequence
  Sphingomonas melonis Fr1
ggaacgcagcgccgcgtccgaacgtt

SEQ ID NO: 14 - DNA, 25 bp, Artificial Sequence
  Sphingomonas melonis Fr1
ggagcattcggcgcgcatcgacgtt

SEQ ID NO: 15 - DNA, 25 bp, Artificial Sequence
  Sphingomonas melonis Fr1
gcaacccggcggtggaggccgcttt

SEQ ID NO: 16 - DNA, 26 bp, Artificial Sequence
  Sphingomonas melonis Fr1
ggcacgcccgacgacctccacccgct

SEQ ID NO: 17 - DNA, 26 bp, Artificial Sequence
  Sphingomonas melonis Fr1
ggaacggatgccagcagcatcccggt

SEQ ID NO: 18 - DNA, 25 bp, Artificial Sequence
  Sphingomonas melonis Fr1
ggaacgaatggcccgaccgatcatt

SEQ ID NO: 19 - DNA, 25 bp, Artificial Sequence
  Sphingomonas melonis Fr1
ggaaccaacgaacgcgactttggtt

SEQ ID NO: 20 - DNA, 25 bp, Artificial Sequence
  Sphingomonas melonis Fr1
ggacccaaaacgggtttcgggggtt

SEQ ID NO: 21 - DNA, 25 bp, Artificial Sequence
  Sphingomonas melonis Fr1
gcatccatttggaacacgggtcgtt

SEQ ID NO: 22 - DNA, 26 bp, Artificial Sequence
  Sphingomonas melonis Fr1
ggaaccggcgcggccacaaggcggtt

SEQ ID NO: 23 - DNA, 27 bp, Artificial Sequence
  Sphingomonas melonis Fr1
cgaactcgtggcccgcgtcagcgcggt

SEQ ID NO: 24 - DNA, 25 bp, Artificial Sequence
  Sphingomonas melonis Fr1
gcgacgaacgcatccgccgccgatt

SEQ ID NO: 25 - DNA, 26 bp, Artificial Sequence
  Sphingomonas melonis Fr1
gcaaccgccggggcctgtcttgcgtt

SEQ ID NO: 26 - DNA, 25 bp, Artificial Sequence
  Sphingomonas melonis Fr1
ggaacaactccgccacgccggagta

SEQ ID NO: 27 - DNA, 25 bp, Artificial Sequence
  Sphingomonas melonis Fr1
ggaaccccctcgcccctcctccgtt

SEQ ID NO: 28 - DNA, 25 bp, Artificial Sequence
  Sphingomonas melonis Fr1
ggatcgcgtgccgggcctgcgcgtt

SEQ ID NO: 29 - DNA, 26 bp, Artificial Sequence
  Sphingomonas melonis Fr1
ggaaccgtgcgcgtcgcctgggcatt

SEQ ID NO: 30 - DNA, 25 bp, Artificial Sequence
  Sphingomonas melonis Fr1
gcaaccaacccccgatgcgctcgtt

SEQ ID NO: 31 - DNA, 25 bp, Artificial Sequence
  Sphingomonas melonis Fr1
ggaacccttaccgatgcacgccgtt

SEQ ID NO: 32 - DNA, 25 bp, Artificial Sequence
  Sphingomonas melonis Fr1
ggaacttcggtccaaatggtgcgtt

SEQ ID NO: 33 - DNA, 25 bp, Artificial Sequence
  Sphingomonas melonis Fr1
ggaaccgccgagcggatcgcgcgtt

SEQ ID NO: 34 - DNA, 25 bp, Artificial Sequence
  Sphingomonas melonis Fr1
gatacgcccgcgatcccaacgcgtt

SEQ ID NO: 35 - DNA, 25 bp, Artificial Sequence
  Sphingomonas melonis Fr1
cgatcggctgcggcatctgcgcatt

SEQ ID NO: 36 - DNA, 25 bp, Artificial Sequence
  Sphingomonas melonis Fr1
ggcacggcgctgtgcgacgccggct

SEQ ID NO: 37 - DNA, 29 bp, Artificial Sequence
  Sphingomonas melonis Fr1
gaaacgcccgcccagcccagccagccggt

SEQ ID NO: 38 - DNA, 25 bp, Artificial Sequence
  Sphingomonas melonis Fr1
gcaacccgctgggctgcgggacgtt

SEQ ID NO: 39 - DNA, 25 bp, Artificial Sequence
  Sphingomonas melonis Fr1
ggatcaaccgcgggggacggccgtt

SEQ ID NO: 40 - DNA, 27 bp, Artificial Sequence
  Sphingomonas melonis Fr1
gcgacgcgctggcgccatcgcgccgct

SEQ ID NO: 41 - DNA, 25 bp, Artificial Sequence
  Sphingomonas melonis Fr1
ggaacggctccattcctgaccagtt

SEQ ID NO: 42 - DNA, 25 bp, Artificial Sequence
  Sphingomonas melonis Fr1
gcaacccgcccgcctcggctgcgtt

SEQ ID NO: 43 - DNA, 26 bp, Artificial Sequence
  Sphingomonas melonis Fr1
ggaaccggtatcgcctgcgtaacgtt

SEQ ID NO: 44 - DNA, 25 bp, Artificial Sequence
  Sphingomonas melonis Fr1
ggtgcgccggcgtgcgctcgccgct

SEQ ID NO: 45 - DNA, 26 bp, Artificial Sequence
  Sphingomonas melonis Fr1
cgaaccgtttcccccgtccgctcgtt

SEQ ID NO: 46 - DNA, 28 bp, Artificial Sequence
  Sphingomonas melonis Fr1
ggaacagatgctggcccagcgcctcgtc

SEQ ID NO: 47 - DNA, 25 bp, Artificial Sequence
  Sphingomonas melonis Fr1
gccacgccccggcctgctgcgcgtg

SEQ ID NO: 48 - DNA, 25 bp, Artificial Sequence
  Sphingomonas melonis Fr1
ggctcgaagcgctcgccgaccggtt

SEQ ID NO: 49 - DNA, 25 bp, Artificial Sequence
  Sphingomonas melonis Fr1
gaaaccttcctgcgcgtcggcggct

SEQ ID NO: 50 - DNA, 24 bp, Artificial Sequence
  Sphingomonas melonis Fr1
ggtcgtcggcgacgccggccggtt